(19) United States
(12) Patent Application Publication
(10) Pub. No.: US 2019/0275324 A1
(43) Pub. Date: Sep. 12, 2019

(54) METHOD FOR TREATING OR PREVENTING A SICKLE-CELL DISEASE AND SYMPTOMS THEREOF

(71) Applicant: DIAVEIN, SAINT FELIX (FR)

(72) Inventors: ETIENNE L'HERMITE, LA CONVERSION (CH); JACQUES L'HERMITE, LA CIOTAT (FR)

(21) Appl. No.: 16/331,210

(22) PCT Filed: Sep. 7, 2017
(86) PCT No.: PCT/EP2017/072408

§ 371 Date: Mar. 7, 2019

Related U.S. Application Data
(60) Provisional application No. 62/384,736, filed on Sep. 8, 2016.
(60) Provisional application No. 62/455,785, filed on Feb. 7, 2017.

(51) Int. Cl. A61N 1/36; A61P 29/00; A61P 7/00

(52) U.S. Cl. CPC A61N 1/36021; A61N 1/36182; A61P 7/00; A61P 29/00

(57) ABSTRACT

The invention relates to the prevention and the treatment of a hemoglobinopathy and symptoms thereof by vascular electrical stimulation therapy (VEST). Preferred hemoglobinopathies are sickle-cell diseases. In a particular embodiment, the invention relates to the prevention and the treatment of vaso-occlusive crisis in patients suffering from a sickle cell disease, by VEST. VEST may be combined with administration of a NSAID to alleviate vaso-occlusive crisis. The invention also relates to the long-term treatment of a symptom associated with a sickle cell disease by VEST.

### FIELD OF THE INVENTION

The invention relates to the prevention and the treatment of a hemoglobinopathy and symptoms thereof. Hemoglobinopathies of particular interest are sickle-cell diseases. In particular, the invention relates to the treatment of vaso-occlusive crisis in patients suffering from a sickle cell disease. The invention also relates to the long-term treatment of a symptom associated with a sickle-cell disease.

### BACKGROUND OF THE INVENTION

Sickle-cell disease (SCD) refers to a group of common genetic disorders which affect hemoglobin, the molecule in red blood cells (erythrocytes) that delivers oxygen to cells throughout the body. Sickle cell disease predominantly affects people from equatorial African, Mediterranean, Indian and Middle Eastern lineage. Approximately 5% of the world's population carries trait genes for haemoglobin disorders and over 300 000 babies with severe haemoglobin disorders are born each year. Around 90 000 to 100 000 people suffer from a sickle cell disease in the US. People with a sickle cell disorder inherit two abnormal hemoglobin genes, one from each parent. In sickle cell diseases, at least one of the two abnormal genes encodes for an atypical hemoglobin molecules, called hemoglobin S (HbS). This abnormal hemoglobin S is caused by a single substitution in the gene encoding for human β-globin subunit, which results in the substitution of valine for glutamic acid on at the 6^{th} position of β-globin chain (β^{S}-globin). This mutation allows HbS to polymerize when deoxygenated. The intracellular polymerization of HbS increases the rigidity of erythrocytes and distorts their cell membrane, leading to sickled erythrocytes. Sickled erythrocytes have impaired plasticity and rheological properties and show altered cell adhesion to vascular endothelium. The other abnormal hemoglobin genes which can be associated with HbS in sickle cell diseases encompass Hemogloin C (HbC) gene, Hemoglobin E (HbE), and mutated HBB gene associated with β-thalassemia phenotype. Hemoglobin C (HbC) gene encodes a mutant of β-globin submit in which the glutamic acid at position 6 is substituted with a lysine residue. Such mutation in the β-globin chain alters the plasticity of red blood cells. Hemoglobin E results from a G→A substitution in codon 26 of the β globin gene, which produces a structurally abnormal haemoglobin as well as activates a cryptic splice site, resulting in abnormal messenger RNA (mRNA) processing. Hence, haemoglobin E is synthesized at a reduced rate, and behaves like a mild form of β-thalassaemia (Olivieri et al., Indian J Med Res, 2011, 134(4):522-531). β-thalassemia phenotypes refer to the absence or a reduced production of β-globin. Mutated HBB genes associated with a phenotype characterized by an absence of β-globin refer to beta-zero (β⁰) thalassemia. Mutated HBB genes associated with the production of beta-globin in reduced amounts refer to beta-plus (β⁺) thalassemia. Accordingly, sickle cell diseases encompass the homozygotic HbSS disease (also called sickle-cell anemia) and other sickle genotypes such as HbSC disease (double heterozygote for HbS and HbC), HbS/β⁰ thalassaemia (severe double heterozygote for HbS β⁰ thalassemia), HbS/β⁺ thalassaemia (double heterozygote for HbS and β⁺ thalassemia), HbS/HbE syndrome (double heterozygote for HbS and HbE) (Stuart and Nagel, the Lancet, 2004, 364, 1343-1357).

The phenotype of sickle cell disease is due to the presence of HbS gene and can be also modulated by pleiotropic genes, unlinked with the β-globin locus. The pleiotropic genes can either ameliorate or exacerbate the HbS phenotype, whereby the severity of sickle cell disease varies greatly between individuals. The clinical manifestations of sickle cell disease are various and encompass vaso-occlusive crisis, anemia, priapism, splenic sequestration crisis, acute chest syndrome, aplastic crisis, haemolytic crisis, stroke, necrosis, acute respiratory distress syndromes and an increased susceptibility to infections. Sickle cell diseases can lead to chronic dysfunctions of organs such as eyes, kidneys, lungs, brain, liver, heart, bones, joints and spleen, which are associated with pronounced mortality and morbidity.

Vaso-occlusion is one of the hallmarks and major complications of sickle cell disease resulting in acute debilitating episodic and recurrent pain episodes. It is also one of the earliest manifestations of sickle-cell disease (SCD), often beginning in infancy and responsible for 90% of hospitalizations in children with SCD. Vaso-occlusion contributes to infection, acute chest syndrome, splenic sequestration, stroke, acute and chronic multisystem organ damage, and shortened life expectancy. It was originally thought that vaso-occlusive crisis resulted from microcirculatory obstruction by poorly deformable sickled red blood cells. The actual pathophysiology of vaso-occlusive crisis is indeed much complex and not fully understood. It involves a plurality of factors including abnormal interactions between poorly deformable stress reticulocytes and vascular endothelium, dysregulation of vascular tone; activation of monocytes, upregulation of adhesion molecules and pro-coagulant factors, hemostasis alteration; and reperfusion injury. These vascular disturbances increase red blood cell transit time, prolonging deoxygenation, which promotes further sickling and vaso-occlusion.

Vaso-occlusive crisis can be also observed in patients suffering from a hemoglobinopathy not associated with HbS gene. Such hemoglobinopathies encompass, for instance HbE/β⁺ and HbE/β° thalassaemia.

The treatment of vaso-occlusion crisis is mainly symptomatic and generally requires the hospitalization of the patient. Painful vaso-occlusion crises are treated by hydration, blood transfusion and/or pain management based on the regular administration of nonsteroidal anti-inflammatory drugs (NSAID), non-opioid and/or opioid analgesics. The administration of NSAID was shown to be poorly effective to manage of vaso-occlusive crisis.

Alternative treatments have been recently proposed to the management of vaso-occlusion crisis. For instance, Rivipansel (GMI-1070), a pan-selectin antagonist developed by GlycoMimetics, Inc, is under phase III clinical trial for the treatment of vaso-occlusive crisis associated with sickle cell disease.

As of today, there is still no effective, in particular no mechanism-based treatment, approved for treating vaso-occlusive crisis of sickle-cell disease.

There is thus a need for alternative treatments of vaso-occlusive crisis in patients suffering from a sickle-cell disease.

### SUMMARY OF THE INVENTION

The invention relates to a method for treating or preventing vaso-occlusive crisis in a patient suffering from a hemoglobinopathy, preferably a sickle-cell disease, which comprises subjecting the patient to vascular electrical stimulation therapy (VEST). The invention also relates to the use of nonsteroidal anti-inflammatory drug (NSAID) in combination with vascular electrical stimulation therapy (VEST) for treating a vaso-occlusive crisis in a patient suffering from a hemoglobinopathy, preferably a sickle-cell disease.

The nonsteroidal anti-inflammatory drug (NSAID) may be an acetic acid derivative, such as diclofenac.

Typically, the electrical periodic current delivered to the patient by VEST in the methods and uses of the invention has a frequency from 0.01 Hz to 15 Hz, preferably from 0.4 Hz to 9 Hz. The periodic current may be composed of electric pulses, the duration of each pulse being of at least 0.5 ms with a rise duration (T₁) of at least 0.25 ms and a decrease duration (T₂) of at least 0.25 ms. The electrical pulses may be unidirectional positive pulses, or unidirectional negative pulses. Alternatively, the electrical current may be composed of streams of pulses which are alternatively negative or positive. In some embodiments, the electrical current has at least one of the following features:
the extinction duration (T₁) of the pulse is from 0.2 to 5-fold the rise duration (T₂) of the pulse,
The rise duration (T₁) is of at least 0.25 ms and the decrease duration (T₂) is of at least 0.25 ms
the peak amplitude of the pulse is from −130 V to 130 V,
the base width of the pulse is from 0.5 ms to 30 ms, and
the period of the electrical current is from 100 ms to 5000 ms.

In some additional embodiments, the pulse of the electrical periodic current has an exponential-type waveform. The electrical periodic current is preferably administered to the patient by transcutaneous route, with a medical device for electrotherapy, comprising at least two electrodes disposed on the skin of the patient. In some particular embodiment, the medical device for electrotherapy comprises at least four electrodes, at least one electrode being disposed on each wrist and each calf of the patients. The patient may be subjected to vascular electrical stimulation therapy after being rehydrated and/or administered with drug(s) selected from anti-inflammatory drugs and analgesics. In some embodiment, the patient is subjected to at least one VEST session per day, which is repeated during at least two consecutive days. In some other embodiments, the patient is subjected to one to four VEST sessions per day, each VEST session lasting from about 10 min to about one hour. In some embodiments, the patient suffers from a hemoglobinopathy selected from sickle-cell anemia, HbSC disease, HbS/HbE syndrome, HbA/HbS genotype, HbE/β⁺ thalassaemia, HbE/β° thalassaemia, HbS/β⁰ thalassaemia, and HbS/β⁰ thalassaemia. In some other embodiments, the patient suffers from a sickle disease. The sickle cell disease may be selected from the group consisting of sickle-cell anemia, HbSC disease, HbS/β⁰ thalassaemia, and HbS/β⁺ thalassaemia.

In another aspect, the invention relates to a method for improving the general condition, or for treating (i.e. delaying the onset of) one or several symptoms associated with a hemoglobinopathy, preferably a sickle cell disease in a patient, wherein the patient is subjected to a long-term treatment with VEST. VEST may be chronically administered to the patient during at least 3 months, Typically, the patient is subjected to one or two VEST treatment of 10 min to 1 hour, every day. In some embodiments, the symptom of the sickle-cell disease is selected from anemia, chronic pain, chronic fatigue, joint pain, rheumatism, breathlessness, priapism, hand-foot syndrome, splenic sequestration, and combinations thereof. In preferred embodiments, the current administered to the patient is as described previously for the treatment or the prevention of vaso-occlusive crisis associated with a hemoglobinopathy, preferably a sickle-cell disease.

### FIGURES

FIG. 1A shows one pulse waveform according to the invention comprising an exponential rise part and an exponential decrease part. Uₘₐₓ: refers to the amplitude of the pulse in V, T₀ refers to the base width of the pulse (namely the duration of the pulse), T₁ refers to the rise duration of the pulse and T₂ refers to the extinction (decrease) duration of the pulse. τ₁ and τ₂ are the time constants of the exponential curves. X-axis: time (t) in ms. Y-axis: voltage (V) in volts.

FIG. 1B shows an example of positive and negative electrical pulses according to the invention for an electrical resistance of 2 kΩ. The pulses have a base width of 2.8 ms, and an amplitude of +22 V or −22 V. X-axis: time (t) in ms. Y-axis: voltage (V) in volts.

FIG. 1C shows an example of positive and negative electrical pulses according to the invention for an electrical resistance of 2 kΩ. The pulses have a base width of 3.2 ms, and an amplitude of +62 V or −62 V. X-axis: time (t) in ms. Y-axis: voltage (V) in volts.

FIG. 2 and FIG. 3 show the results of the clinical study described in Example 2. FIG. 2 shows the mean pain score in visual analog scale (VAS) for pain, over time, in patients treated by VEST and in patients treated with morphine. FIG. 3 shows the mean pain score over time in pediatrics patients and in adult patients.

FIG. 4 shows the visual analog scale (VAS) for pain used for assessing pain in the clinical studies.

FIG. 5 shows the results of the clinical study described in Example 5, namely the mean pain score in visual analog scale (VAS), over time, in patients treated by VEST, in patients treated with VEST and NSAID and in patients treated with NSAID only.

### DETAILED DESCRIPTION OF THE INVENTION

The Applicant demonstrated that vascular electrical stimulation therapy is effective in the management of acute and painful vaso-occlusive crisis in patients with sickle-cell disease. As illustrated in the case reports described in Example 2 and confirmed in the clinical study of Example 3, VEST enables to reduce the duration and/or the intensity of the vaso-occlusive crisis, the intensity of the pain, the length of hospital stay and the use of opioid analgesics for the management of pain. Noteworthy, VEST was shown to be significantly more effective than morphine to alleviate pain in patients with acute vaso-occlusive crisis: In patients treated with VEST, the mean pain score fell below 2 cm in VAS after 4 hours, while the mean pain score remains higher than 6 cm (severe pain) in patients treated with morphine.

VEST is also effective as a prophylactic or maintenance treatment in order to prevent the occurrence of subsequent vaso-occlusive crisis and/or decrease their intensity in a patient with a sickle-cell disease. Furthermore, VEST is safe, as it is not associated with any adverse effect or iatrogenic risk.

Without to be bound by any theory, the Applicant believes that the efficiency of VEST to treat vaso-occlusive crisis and alleviate pain results from specific effects exerted on the vascular system, including blood flow increase, and vascular dilatation. VEST also promotes the release of fibrinolytic and antithrombotic factors.

The Applicant also showed that the combination of VEST with nonsteroidal anti-inflammatory drug (NSAID) can improve and even potentiate the management of the vaso-occlusive crisis and pain in a patient suffering from a sickle-cell disease.

Thus, according to a first aspect, the invention relates to a method for treating or preventing a vaso-occlusive crisis in a patient suffering from a hemoglobinopathy, preferably a sickle-cell disease, said method comprising subjecting the patient to a vascular electrical stimulation therapy. The invention also relates to a method for alleviating pain in a patient suffering from a vaso-occlusive crisis, preferably a severe vaso-occlusive crisis, associated with a hemoglobinopathy, preferably a sickle-cell disease. The invention also relates to the use of vascular electrical stimulation therapy for treating or preventing a vaso-occlusive crisis in a patient suffering from a hemoglobinopathy, preferably a sickle-cell disease.

A further object of the invention is the use of a NSAID in combination with VEST for the treatment or the prevention of vaso-occlusive crisis in a patient suffering from a sickle-cell disease. The invention also relates to the use of a NSAID in combination with VEST for the management of pain associated with a vaso-occlusive crisis in a patient suffering from a hemoglobinopathy, preferably a sickle-cell disease.

As used herein, “hemoglobinopathy” refers to inherited disorders resulting in an abnormal structure of at least one chain of a hemoglobin. Abnormal hemoglobin genes encompass, without being limited to, HbS, HbC, HbE HbD, Hb O-Arab and Hb G-Philadelphia. Hemoglobinopathies encompass, without being limited to, sickle cell diseases as well as HbE/β⁺ and HbE/β° thalassaemia.

In a particular embodiment, the patient carries HbS gene and is thus homozygote or heterozygote for HbS gene. In some embodiments, the patient is of the genotype HbA/HbS, HbA corresponding to the gene encoding for normal hemoglobin.

In a preferred embodiment of the invention, the patient suffers from a sickle-cell disease. As used herein, “sickle-cell disease” refers to inherited disorders associated with HbS gene. Sickle cell disease is characterized by the presence in the patient's blood, of misshapen blood red cells, namely sickled blood red cells. Typically, sickled blood red cells account for at least 50% of blood cells present in the patient's blood. Sickle-cell diseases encompass, without being limited to, homozygotic HbSS disease (also called sickle-cell anemia), HbSC disease, HbS/β⁰ thalassaemia, HbS/β⁺ thalassaemia, HbS/hereditary persistence fetal Hb (S/HPHP), HbS/HbE syndrome, haemoglobin SD, Punjab disease, haemoglobin SO Arab disease, and others.

In some embodiments, the patient suffers from a hemoglobinopathy selected from sickle-cell anemia, HbSC disease, HbS/HbE syndrome, HbA/HbS genotype, HbE/β⁺ thalassaemia, HbE/β° thalassaemia, HbS/β° thalassaemia, and HbS/β⁺ thalassaemia.

In some embodiments, the patients suffer from a sickle cell disease selected from sickle-cell anemia, HbSC disease, HbS/β⁰ thalassaemia, and HbS/β⁺ thalassaemia.

A preferred sickle-cell disease is sickle-cell anemia.

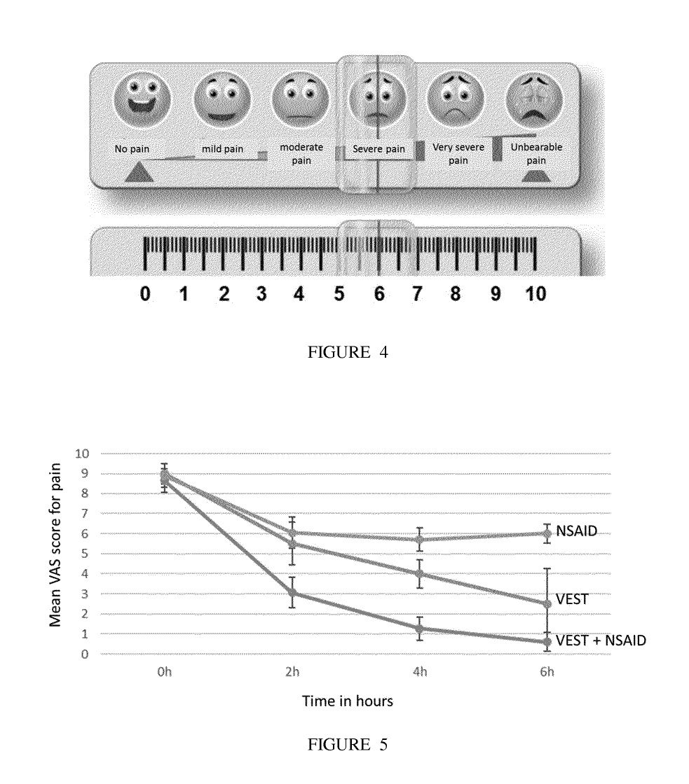

As used herein, “Vaso-occlusive crisis” or “Vaso-occlusion crisis” refers to vaso-occlusive crisis associated or caused by a hemoglobinopathy, preferably a sickle-cell disease. Vaso-occlusion refers to episodic microvessel occlusion at one or many sites of the patient with sickle-cell disease. Vaso-occlusion can affect any part of the body of the patient, in particular bones (long bones, ribs, sternum, pelvis), hands and feet, in particular in children, and intestines (by vaso-occlusion of mesenteric vessels). Vaso-occlusion is generally associated with acute pain, inflammation and ischemia. The pain is generally very intense and can take different forms depending on the affected organ: bone pain, abdominal pain, arthralgia, osteonecrosis, pulmonary infract, hand-foot syndrome. Pain can be assessed with a 10-cm visual analog scale (VAS) for pain, for instance as shown in FIG. 4. Vaso-occlusive crisis is generally associated with a pain score of at least 4 cm in VAS for pain. A severe vaso-occlusive crisis refers to a vaso-occlusive crisis with a pain score of at least 6 cm in VAS for pain.

As used herein, a NSAID refers to a drug class of non-steroidal compounds able to reduce pain and to decrease fever and inflammation. These compounds can be classified depending on their chemical structure and their capacity to inhibit a cyclooxygenase. Cyclooxygenases 1 and 2 are the two main isoforms. Most NSAIDs are mixed inhibitors of the two isoforms. Five categories of mixed inhibitors may be defined: (1) propionic acid derivatives, (2) acetic acid derivatives; (3) fenamic acid derivatives; (4) salicylate derivatives; and (5) enolic acid derivatives (oxicams).

Acetic acid derivatives encompass diclofenac, indomethacin, tolmetin, sulindac, etodolac, ketorolac, aceclofenac, and nabumetone.

Propionic acid derivatives encompass ibuprofen, dexibuprofen, naproxen, fenoprofen, ketoprofen, dexketoprofen, flurbiprofen, oxaprozin and loxoprofen.

Oxicams encompass piroxicam, meloxicam, tenoxicam, droxicam, lornoxicam, and phenylbutazone.

Fenamic acid derivatives encompass mefenamic acid and meclofenamic acid.

Salicylate derivatives encompass acetylsalicylic acid and diflunisal.

NSAIDs also encompass selective COX-2 inhibitors such as celecoxib, rofecoxib, and parecoxib, preferably celecoxib.

In some embodiments, the NSAID is selected from propionic acid derivatives such as ketoprofen and acetic acid derivatives such as diclofenac.

Preferred NSAIDs according to the invention are acetic acid derivatives. A preferred NSAID is diclofenac.

The NSAID can be administered to the patient by any conventional routes such as the oral, rectal, or the parenteral route, for example by intravenous, intracutaneous or intradermal injection. In some embodiment, the NSAID is administered by oral route or intravenous route.

The dosage and the frequency of administration depends on the NSAID to be used, the severity of the vaso-occlusive crisis and the intensity of pain. The dosage administered to the patients is generally from 1 mg to 800 mg, such as from 10 mg to 400 mg. The administration can be repeated until the pain and/or the vaso-occlusive crisis are managed, for instance every 2, 4, 6 or 8 hours.

As used herein, “vascular electrical stimulation therapy (VEST)” refers to a therapeutic treatment based on the external and non-invasive administration of low-frequency electrical pulses able to stimulate vascular smooth muscle and/or vascular endothelium in the patient. As used herein, a low frequency electrical pulses refer to an electrical periodic signal having a frequency from 0.01 to 15 Hz, preferably from 0.1 to 10 Hz such as a frequency from 0.4 to 8.5 Hz, or from 0.5 Hz to 4 Hz. An appropriate frequency is for instance 1.6±0.2 Hz.

It should be noted that “vascular therapy electrical stimulation therapy” is distinct from “transcutaneous electrical nerve stimulation (TENS)” in terms of delivered electrical pulses and the stimulated tissue. In the case of VEST, the target tissue is the vascular system, in particular vascular smooth muscle through the sympathetic nervous system and/or the vascular endothelium. By contrast, in TENS, the target tissue is the skeletal striated muscles through the somatic nervous system.

An electrical current appropriate to provide VEST in the patient according to the invention refers to a periodic current having a frequency and a pulse waveform able to stimulate the smooth muscle and/or the endothelium of the vascular tissue.

In some embodiments, the electrical signal does not induce a significant stimulation of motor and/or sensitive nerves.

The periodic electrical current delivered to the patient can be composed of unidirectional positive pulses or, unidirectional negative pulses. Alternatively the periodic current is in the form of streams of pulses that are alternatively positive or negative. In some embodiments, the current delivered to the patient is composed of identical pulses.

Without to be bound by any theory, the Applicant is of the opinion that alternating unidirectional positive pulses groups and unidirectional negative pulses groups may improve the efficacy in VEST.

In preferred embodiments, the electrical current delivered to the patient consists in alternatively positive and negative streams of pulses. Each stream comprises from 2 to 20, for instance from 6 to 10 identical pulses. As an example, the periodic electrical current administered to the patient may be composed of the repetition of 8 positive pulses followed by 8 negative pulses.

The waveform of the pulses may be of any type with proviso that the duration of the pulse is of at least 0.5 ms with a rise duration (T₁) of at least 0.25 ms and a decrease duration (T₂) of at least 0.25 ms. As used herein, T₁ refers to the duration of the rise part of the pulse from the baseline to the pulse maximum peak. T₂ refers to the duration of the decrease part of the pulse from the pulse maximum peak to the baseline. Thus, the waveform of the pulses is not squared or rectangular.

In some embodiments, the electrical periodic current delivered to the patient is characterized by one or several of, preferably all, the following features:
the pulse has a duration (namely a base width—T₀) from 0.5 ms to 30 ms. Preferably the pulse duration is from 0.5 to 5 ms, for instance from 1.5 ms to 4 ms, or from 2.0 ms to 3.5 ms, such as 2.5 ms.
the extinction (or decrease) duration (T₂) of the pulse is from 0.2 to 5-fold, preferably from 0.5 to 1.5-fold the rise duration (T₁) of the pulse. Preferably the two durations are equal.
The rise duration (T₁) of the pulse is at least 0.25 ms, preferably at least 0.5 ms, such as at least 0.6, 0.8, and 1.0 ms.
The decrease duration (T₂) of the pulse is at least 0.25 ms, preferably at least 0.5 ms, such as at least 0.6, 0.8, and 1.0 ms.
the peak amplitude of the pulse (Umax) is from −130 V to 0 V or from 0 V to 130 V, preferably from −100 V to −10 V or 10 V to 100 V, such as from −10 V to −60 V or from 10 V to 60 V.
the period of the electrical current is from 100 ms to 5 000 ms, for instance from 120 ms to 2200 ms such as a period from 250 ms to 2000 ms. For instance, the period of the electrical current may be about 560 ms to 720 ms such as about 625 ms

For instance, an appropriate electrical periodic current to provide VEST may be characterized by:
a pulse duration from 1.5 ms to 4.0 ms
a ratio of T₂ to T₁ from 0.5 to 1.5,
a T₁ of 0.5 ms to 2.0 ms
a T₂ of 0.5 ms to 2.0 ms
a Umax from −60 V to −10 V or from 10 V to 60 V, and
a period from 560 ms to 720 ms.

In some embodiments, Umax is from 30 V to 60 V or from −60 V to −30 V.

In some additional or alternate embodiments, the maximum intensity of the current (namely the intensity at the pulse peak) is at most 100 mA. Preferably, the peak intensity is at most 90 mA, preferably at most 80 mA. Typically the peak intensity may range from 0.5 mA to 60 mA.

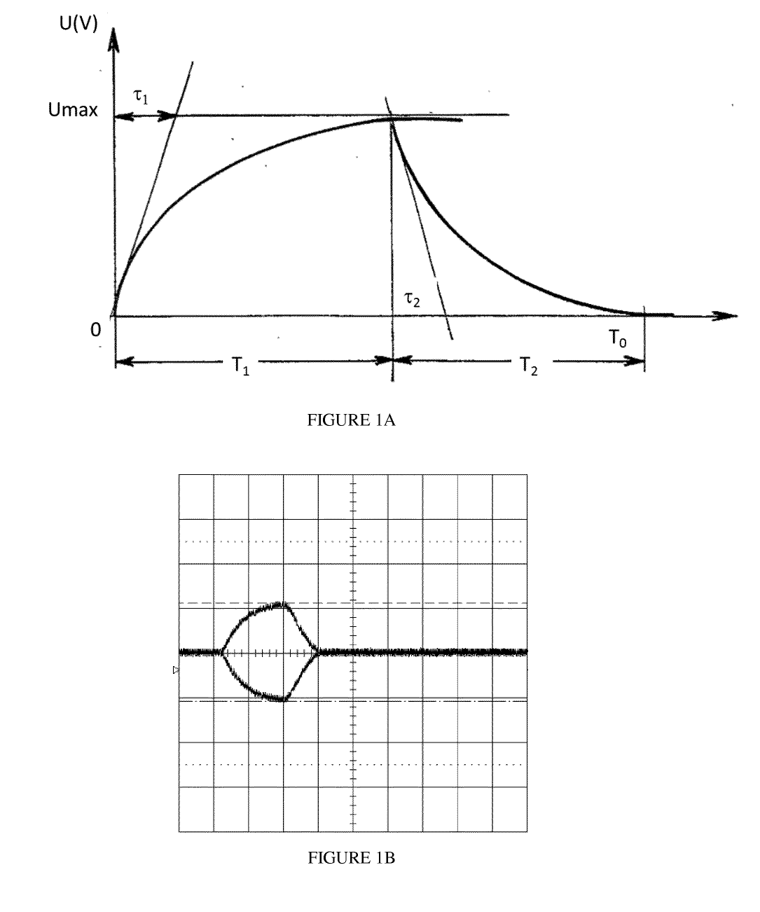

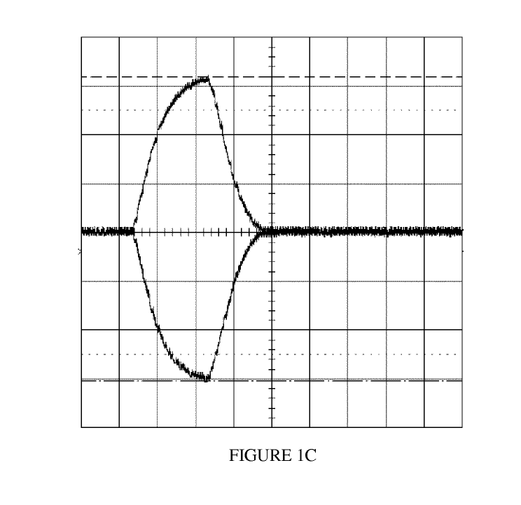

In a preferred embodiment, the waveform of the pulse is of the exponential type. This means that the pulse comprises an increase part and a decrease part which correspond to exponential curves. An example of a pulse of the exponential type is shown in FIG. 1A. In a preferred embodiment, the electrical pulses of the current administered to the patient has:
an rise part of formula (I):

$$U = U max ( 1 - {e}^{- \frac{t}{τ 1}} ) \qquad ( I )$$

a decrease (or extinction) part of formula (II)

$$U = U max ( 1 - {e}^{- \frac{t - T 1}{τ 2}} )$$

wherein U refers to voltage, t refers to time, Umax is the amplitude peak of the pulse, T₁ is the duration of the rise part of the pulse and τ₁ and τ₂ refer to the time constants. In some embodiments, τ₁ is equal to τ₂. In certain embodiments, τ₁ and τ₂ is from about 0.1 to about 0.8 ms.

In the context of the invention, VEST may be performed with any medical device for delivering an electrical current able to stimulate the human vascular smooth muscle and/or the vascular endothelium in the patient though electrodes disposed on specific body area. The medical device comprises at least two electrodes, because at least two electrodes should be positioned to enable the flow of electrical current through the patient's body. The administration of the electrical pulses is preferably performed by transcutaneous route. The medical device comprises at least two electrode pads which are positioned on appropriate skin area of the patient's body.

Typically, the electrodes are positioned on the limbs of the patient, regardless the site of the vaso-occlusive crisis. In some preferred embodiments, the medical device comprises four electrodes. Preferably, an electrode is positioned on each calf (or plantar arch) and each wrist (or palms) of the patient. Without to be bound by any theory, the Applicant believes that such positioning of the electrode pads may promote an effective and global stimulation of the vascular system in the patient. Alternatively, an electrode may be positioned on each calf and on the chest of the patient.

Any medical electrotherapy device can be used in the context of the invention, with proviso that the device can deliver an appropriate electrical current as defined above. For instance, the medical device may be as described in EP 0 137 007 or in U.S. Pat. No. 5,725,563, the disclosure of which being incorporated herein by reference. An appropriate medical electrotherapy device is also available on the market, namely DIAVEIN, marketed by CT Sciences SA, DIAVEIN Sarl, Typically, the medical device for administering the vascular electrical stimulation according to the invention comprises an electrical current generator unit including (i) a mean for generating an electrical pulse current and (ii) a mean for controlling the generated electrical current, said electrical current-generating unit being connected to at least two electrodes or electrode pads adapted to be placed on the patient, preferably on the skin, and delivering electrical stimulation to said patient. The mean for controlling the generated electrical current may enable to adjust the intensity, the shape, the frequency and/or the voltage of said current. Said controlling mean may comprise a selector unit enabling to select the frequency, the pulse waveform, the pulse duration, the pulse amplitude, the intensity and/or the energy of the current, and/or preset programs able to control the delivery of preset electrical currents to the patient.

Optionally, the medical device may comprise a measuring unit for measuring the patient-dependent electrical parameters, such as impedance, and a processing unit in communication with said measuring unit and the electrical current generator unit, whereby the electrical current delivered to the patient can be optionally adjusted depending on the measured patient-dependent electrical parameters.

The electrical current to deliver to the patient, may be adjusted depending on the therapeutic effect to achieve (namely the prevention or the treatment of a vaso-occlusive crisis), the severity of the vaso-occlusive crisis to be treated and the sensitivity of the patient to the electrical stimulation

In particular, the pulse peak amplitude (Umax) may vary from one patient to another, depending on the patient impedance and the patient sensitivity to electrical stimulation. The patient impedance may vary from 1 kΩ to 4 kΩ. The higher impedance, the higher Umax. For instance, Umax of the electrical pulse is typically less than 50V for an impedance of 2 kΩ measured between the electrodes. Preferably, a therapeutic electrical current corresponds to an electrical current able to induce a small muscle tremor at each electrode disposed on the patient. The physician may determine the appropriate voltage of the current to deliver to the patient by gradually increasing the voltage of the electrical current while observing the reaction of the patient. At the beginning of the treatment, the physician may gradually increase the intensity of the electrical stimulations until the patient feels them. Then, the physician may let the patient subjected to said current during few minutes, for instance 10 minutes, to check that the patient does not feel any disorder or significant discomfort. The physician may then increase the stimulation until a very slight muscle tremor is observable at the skin area in the contact with the electrodes. If the patient complains about the stimulation, the physician decreases the energy level so as to obtain an electrical stimulation which is comfortable for the patient while providing a small muscle tremor at the electrodes

At the second session, the physician can adjust the energy level of the electrical stimulation at the level previously identified as providing muscle tremor in patients while being comfortable.

It goes without saying that the invention also relates to the use of an electrical current as defined above or to the use of a medical electrotherapy device as defined above for treating or preventing vaso-occlusive crisis in a patient suffering from a hemoglobinopathy, preferably a sickle-cell disease.

In the methods and uses according to the invention, the regimen of the VEST treatment depends on the therapeutic effect which is sought.

Typically, for the treatment of vaso-occlusive crisis, the patient is subjected to at least one VEST treatment session daily, during at least two consecutive days. The daily duration of VEST depends on the severity of the vaso-occlusive crisis. For a severe vaso-occlusive crisis the patient may be subjected to VEST all day long. For a less severe vaso-occlusive crisis, a total duration of VEST of about 4 hours may be sufficient. In other words the total duration of VEST therapy per day may greatly vary from a patient to another. However, a total VEST duration of at most 12 hours, such as a duration of 2 hours to 8 hours may be sufficient for most of the patients.

Typically, the patient is subjected to one or several VEST sessions per day. Each treatment session may last from few minutes to few hours, typically from 10 minutes to four hours. In some embodiments, the patient is subjected to one to eight treatment sessions daily, each treatment session lasting from 10 minutes to one hour, for instance from 30 min to 45 min. Between two consecutive treatment sessions, the patient is allowed to have a break, for instance of 10 min to one hour. The patient can be treated with VEST at any time of the vaso-occlusive crisis. In some embodiments, the patient is subjected to VEST in the early beginning of the vaso-occlusive crisis. Accordingly, in some embodiments, the patient is treated with VEST within 24 hours, preferably within 12 hours and more preferably within 6 hours from the beginning of the vaso-occlusive crisis. For the treatment of vaso-occlusive crisis, VEST sessions may be repeated during several consecutive days, for instance during 2 to 20 consecutive days. For most of the patients, it may be sufficient to repeat the VEST sessions during 3 to 10 consecutive days to manage the vaso-occlusive crisis. For instance, as described in the Example, three to four hours of VEST treatment daily, during three consecutive days, may be sufficient to significantly alleviate the vaso-occlusive crisis and the associated pain in certain patients. However, longer treatment may be performed to treat severe vaso-occlusive crisis.

Depending on the severity of the vaso-occlusive crisis in the patient, VEST may be combined with drug therapy for pain management, inflammation management and/or hydration. Drug therapy encompasses, without being limited to, administration of opioid analgesic, non-opioid analgesic, anti-inflammatory drugs, such as nonsteroidal anti-inflammatory drugs (NSAIDs). In some embodiments, the patient is subjected to hydration and/or administration of NSAIDs and/or analgesics, in particular opioid drugs, prior to or simultaneously to the first VEST session. Hydration and administration of the drugs can be prolonged until an appropriate management of the pain in the patient. The frequency and the dose of analgesics and/or NSAIDs drugs may be reduced, and even stopped, on the second or the third day of treatment with VEST.

In some embodiment, the patient is subjected to VEST without being administered with a NSAID. In a particular embodiments, the patient is subjected to VEST in combination with a treatment selected from hydration, analgesics and combinations thereof.

In some other embodiments, the patient is subjected to both VEST and NSAID treatments. Once the vaso-occlusive crisis and the associated pain is managed, the patient may be subjected to a maintenance VEST treatment so as to prevent a relapse. The use of analgesics, in particular opioid analgesics, is generally already stopped at that stage. In the maintenance treatment, the same regimen as that used in the therapeutic phase can be performed. Alternatively, the frequency and/or the daily duration of VEST can be decreased during the maintenance treatment. For instance, the patient can be subjected to one to four VEST sessions of 10 min to 1 hour per day. The VEST sessions may be performed every day, three times a week, twice a week, or once a week. The maintenance treatment can last from one week to several months, for instance one to three months.

In the context of the prevention of vaso-occlusive crisis, the frequency and/or the daily duration of VEST may be less than that used in the treatment of vaso-occlusive crisis. For instance, the patient may be subjected to one to four VEST sessions of 10 min to 1 hour a day, once or twice a week. In preferred embodiment of the preventive treatment, the patient is subjected to one or two sessions of 10 min to 1 hour every day. For instance, the patient can be subjected to one or two VEST sessions of 10 min to 30 min, daily (for instance in the morning and/or in the evening) during at least 3 months. The preventive treatment can be performed during several months.

In some embodiments of the preventive treatment, the VEST may be combined with the administration of a NSAID. The NSAID may be administered on demand when the patient experiments a symptom or a disorder announcing a further vaso-occlusive crisis, or chronically for instance weekly or daily. The daily dosage of the NSAID may be less than 800 mg, for instance from 5 mg to 200 mg.

During the VEST sessions, the patient may be in a lying position, in particular in supine position.

The patient may be any human being, of any gender, suffering from a hemoglobinopathy, preferably a sickle-cell disease and who suffers from, or is at risk of suffering from, episodic or chronic vaso-occlusive crises associated with said hemoglobinopathy. The patient may be an adult, an adolescent, a child or an infant. In some embodiments, the patient is a child or an infant of at least 6 months.

Without to be bound by any theory, the Applicant is of the opinion that preventive treatment with VEST may treat or reduce one or several additional symptoms of the sickle-cell disease in the patient. In that respect, the Applicant demonstrated that long-term treatment with VEST in patients suffering from a sickle-cell disease enables to improve their general condition by treating or alleviating one or several sickle-cell disease symptoms such as anemia, chronic pain, chronic fatigue, joint pain, rheumatism, breathlessness, priapism, hand-foot syndrome, splenic sequestration and other ischemia-related disorders.

Without to be bound by any theory, the Applicant is of the opinion that VEST may also prevent or delay the onset or the development of disorders associated with sickle-cell disease such as osteoporosis, osteonecrosis, stroke, renal insufficiency, and acute chest syndrome.

Accordingly, the invention also relates to a method for improving the general condition, or for treating one or several symptoms of a patient suffering from a hemoglobinopathy, preferably a sickle-cell disease, wherein the patient is subjected to a long-term treatment with VEST. The invention also relates to the use of VEST, an electrical current as defined above or an electrotherapy device as defined above for improving the general conditions and/or treating one or several symptoms in a patient suffering from a hemoglobinopathy, preferably a sickle-cell disease.

As used herein, a long-term treatment with VEST, means that VEST is chronically administered to the patient during at least 3 months, preferably during at least 6 months, 12 months and even several years.

Typically, the patient is subjected to a VEST treatment in particular as described above, at least once a month, preferably at least once a week and more preferably at least twice a week and even more preferably every day. In preferred embodiments, the patient is subjected to one or two sessions of 10 min to 1 hour, preferably of 10 min to 30 min, every day.

In some embodiments, the long-term treatment is for treating one or several sickle-cell disease symptoms selected from anemia, chronic pain, chronic fatigue, joint pain, rheumatism, breathlessness, priapism, hand-foot syndrome, and splenic sequestration.

In some embodiments, the long-term treatment with VEST may be combined with the administration of a NSAID. The NSAID may be administered on demand, during a limited period of time, when the patient experiments a symptom or a disorder announcing a further vaso-occlusive crisis. The daily dosage of the NSAID may be less than 800 mg, for instance from 5 mg to 200 mg. The NSAID may be administered during at most 2 weeks, for instance during 1 to 7 days.

The following examples are provided by way of illustration only and not by way of limitation.

### EXAMPLES

### Example 1: Material and Method for Implementing VEST

a. General Protocol of Vascular Electrical Stimulation Therapy (VEST)

The Vascular Electrical Stimulation Therapy was performed by using a conventional medical device for vascular electrical stimulation therapy comprising four electrodes (Diavein). For the relief of acute vaso-occlusive crisis, the duration of the treatment is typically of at least of 3 days. The patients is subjected by a 4 hours treatment by VEST daily. The treatment can be interrupted several times by small breaks, upon patient's request. During the treatment sessions, the patient is in supine position. The electrodes are positioned on each calf (or plantar arch) and each wrist (or palms) of the patient. The clinical dose of electrical stimulation refers to an electrical stimulation sufficient to induce a small muscle tremor at each electrode while being comfortable for the patient. The treatment is carried out very gradually as regards to the increase in energy. At the beginning of the treatment, the physician gradually increases the electrical stimulations until the patient feels them. Then, the physician lets 10 minutes elapse to ensure that the patient feels no disorder or significant side effects. The physician then increases the stimulation until a very slight muscle tremor in tissues located in the contact of the electrodes is observable. If the patient complains about the stimulation, the physician decreases the energy level so as to obtain an electrical stimulation which is comfortable for the patient.

At the second session, the physician can adjust the energy level of the electrical stimulation at the dose identified as providing muscle tremor in patients while being comfortable (clinical dose).

The treatment can be interrupted in case of total remission of the crisis before 72 h.

b. Medical Device and Electrical Current Delivered to the Patient

The medical device used to deliver the Vascular Electrical Stimulation Therapy is a Diavein device commercialized by the company CT Sciences SA (Switzerland). Typically the current delivered to the patient when treating a vaso-occlusive crisis ranges from 40 to 50V at a 1.6 Hz on a 1500 to 2500f? impedance measured between the electrodes. The electrical pulse of the signal has an exponential-type waveform as defined in FIG. 1A. The duration of the pulse is from 2.8 ms to 3.5 ms. The electrical current comprises unidirectional positive pulses group followed by unidirectional negative pulses group.

The VEST protocol and material described in Example 1 are those used in the case reports of Example 2 and in the clinical trial of Example 3.

### Example 2: Preliminary Clinical Study Concerning the Assessment Vascular Electrical Stimulation Therapy (VEST) in the Relief of Vaso-Occlusive Crises Associated with Sickle Cell Disease

Case Report 1

The patient was a 29 year-old man, with sickle cell anemia (HbSS). The patient was hospitalized for an acute pain which was progressive for 48 hours and located on his left arm, without any trauma. The patient was diagnosed for a vaso-occlusive crisis. Temperature: 36.6° C., Blood pressure 130/90.

He was administered with one dose of Profenid® (ketoprofen—1 ampoule) and one dose of Trabar® (Tramadol—1 ampoule), when admitted in the hospital.

The patient was treated with vascular electrical stimulation therapy on the 2^{nd} and the 3^{rd} day of hospitalization. On the 2^{nd} day of hospitalization, the patient was subjected to five 45 minutes-sessions of vascular electrical stimulation therapy. On the 3^{rd} day of hospitalization, the patient was subjected to eight VEST sessions of one hour. The patient left the hospital on the 4^{th} day. The patient was subjected to an additional VEST session on day 6, for preventive purpose.

The VEST sessions relieved the patient of the acute pain. The administration of anti-inflammatory drug (profenid) and opioid drug (Tramadol) was thus stopped, just after the first session of VEST. The vascular electrical stimulation enabled to suppress the acute pain caused by the vaso-occlusive crisis without any further administration of painkiller drugs. No secondary side- or adverse effects of VEST were reported in the patient.

Case Report 2 The patient was a 35 year-old woman with sickle cell anemia. The patient suffered from acute and diffuse arthralgia. She was firstly treated with anti-inflammatory drugs by parenteral route, without any significant relief of the pain, which became intolerable. She was hospitalized and diagnosed for a vaso-occlusive crisis. Temperature: 37.2° C., Blood pressure: 130/90, Heartbeats: 109/min.

Following her admission at the hospital, she was administered with morphine by subcutaneous route, then with Perfalgan® (1 ampoule/12 h) and Profenid® (1 amp/12 h). The patient still suffered from diffuse pains and was administered with Perfalgan® and Acupan®. On the 2^{nd} day of hospitalization, the patient experimented pains in the abdomen, in the back and in the thorax. Trabar® (1 amp.) and Nootropyl® (1 amp. twice a day) were administered for treating these pains. The patient was subjected to a first session of VEST, which improved the general condition of the patient. On the 3^{rd} day, further acute pains appeared in the legs and the arms. The patient was administered with Drazepan® and subcutaneous morphine and treated by VEST (4 sessions of 45 minutes).

On the 4^{th} day, the pains were significantly reduced, with a mild pain located on the foot only. The patient was thus significantly relieved by the VEST session and was able to sleep again. The treatment by VEST enabled to manage the vaso-occlusive crisis and to stop the administration of analgesic drugs. No secondary side- or adverse effects of VEST were reported in the patient.

Case Report 3

The patient was a 23 year-old man suffering from sickle cell anemia (Hbss). The patient was hospitalized for diffuse arthralgia and bilateral chest pain. The patient was agitated with conjunctival and palmar-plantar paleness. Temperature: 37.1° C., Blood pressure 100/70, Heartbeats: 90/min, SPO2: 94%. The patient was diagnosed for vaso-occlusive crisis and anemia.

The patient was administered with 11 mg of Profenid® by intramuscular route and then perfused with a rehydration solution comprising Profenid (100 mg per 24 h). The patient was further administered with Perfalgan®, Acupan®, Trabar® (1 amp.) and then morphine (1 mg per intravenous route). The patient received a blood transfusion and then subjected to 4 VEST sessions of 45 min. The patient was alleviated from pains. Preventive treatments by VEST were repeated daily according to the general protocol, during about 1 month. VEST sessions enabled to alleviate pain and manage the vaso-occlusive crisis. The treatment with painkiller drugs was stopped. The prolonged treatment with VEST prevented subsequent vaso-occlusive crises. No side- or adverse effects of VEST were reported in the patient.

### Example 3: Clinical Study Concerning the Assessment of VEST in the Relief of Pain in Vaso-Occlusive Crisis Associated with Sickle Cell Disease

The objective of the clinical study was to assess the efficacy of VEST to manage pain in adult and paediatric patients with a severe vaso-occlusive crisis associated with sickle cell disease. 17 patients suffering from a sickle-cell disease and experimenting a severe vaso-occlusive crisis, with a pain score of at least 6 in the visual analog scale for pain (VAS pain), were enrolled in the study. The enrolled patients comprised 8 adults (of 19 to 35 years) and 9 children (of 8 to 17 years). 5 patients were of HbSS genotype, 11 patients were of HbSC genotype and the one remaining patient suffered from a sickle-cell disease with an undetermined genotype. The enrolled patients comprised 11 male subjects and 6 female subjects.

After their admission at the hospital, the patients received pain killer (Perfalgan) and Dynapar drug and were subjected to VEST treatment for 4 hours, with two electrodes under each calf and two electrodes under each arm of the patient.

Pain change in each patient was assessed with a 10-cm visual analog scale (VAS) for pain (e.g. as shown in FIG. 4) every two hours, during 6 hours after the beginning of VEST. The resulting mean pain scores were compared with mean pain scores obtained in patients treated with morphine only, shown in Weiner et al.'s study (Weiner et al., JAMA, 2003, 289, 9, 1136-1143).

Results

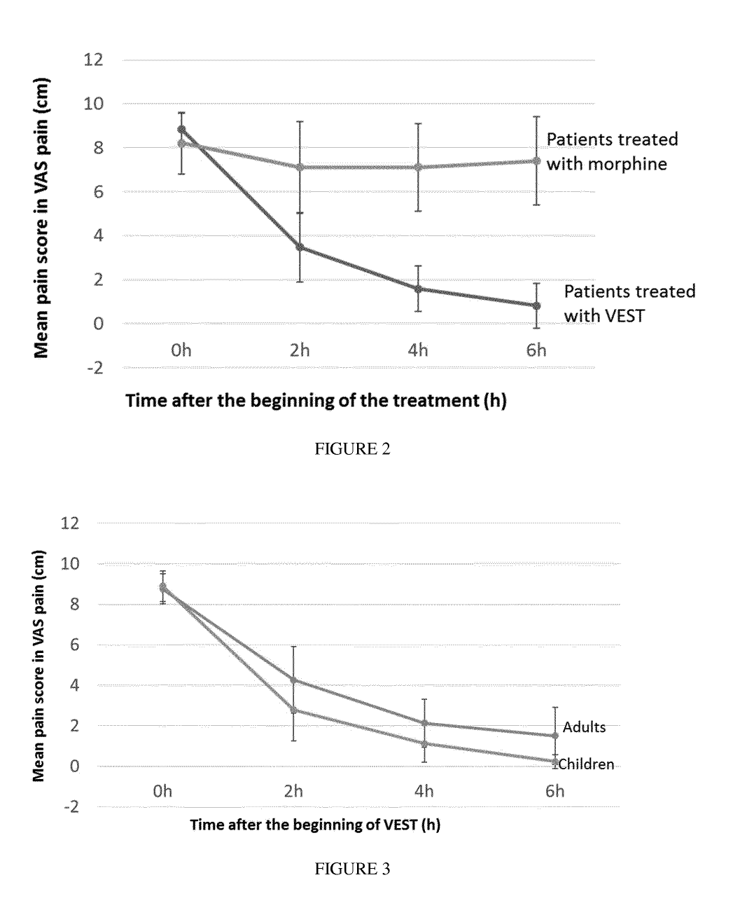

The results of the study are shown in FIG. 2. VEST treatment was effective to manage pain in all patients. The mean pain score in VAS was decreased by 50% within two hours after the beginning of VEST. The mean pain score even fell below 2 cm (mild pain) after 4 hours of treatment. Noteworthy, VEST was able to alleviate pain in all patients without co-administration of opioid drug such as morphine. As shown in FIG. 3, the alleviation of pain was more rapid in paediatrics patients than in adult patients. Noteworthy, the vaso-occlusive crisis was managed within 2 hours in 4 of the 9 children treated with VEST.

As shown in FIG. 2, the magnitude of change in VAS pain score in patients treated with VEST was significantly higher than in patients who received morphine (reference drug), in Weiner study. In other words, VEST therapy was shown to be more effective than morphine to alleviate pain in severe vaso-occlusive crisis associated with sickle cell disease.

### Example 4: Interventional, Prospective, Randomized, Comparative, Single Blind and Mono-Centric Study of Sickle Cell Disease Patients with Vascular Electrical Stimulation Therapy for Treatment of Vaso-Occlusive Crises (VOC)

Objective:

To explore the efficacy and safety of Vascular Electrical Stimulation Therapy (VEST) for the treatment of vaso-occlusive crisis (VOC) in patients with sickle cell disease (SCD).

Main Outcome Measurement:

Change in pain every hour the first 10 hours measured on a 10-cm Visual Analogue Scale (VAS). Secondary outcome measures are duration of hospitalization, blood pressure, platelet aggregation and oxygen saturation.

Study Setting and Population:

20 patients aged from 15 to 30 years with sickle cell anaemia (HbSS), hemoglobin SC (HbSC), or HbS-β-thalassemia (HbS-βthal) who are experiencing uncomplicated severe acute vaso-occlusive crisis (VOC) (score of ≥6 cm on a 10-cm visual analog scale [VAS]) will be enrolled. 10 patients will be subjected to VEST (Group 1). The 10 remaining patients will not be subjected to VEST. Exclusion criteria include VOC concomitant with other acute processes, including but not limited to acute chest syndrome and potential serious infection; transfusion or use of investigational drugs within the last 30 days; smoking more than 0.5 pack per day; and pregnancy. Acute pain crisis is defined as pain in the extremities, chest, abdomen, or back that could not be explained by other complication of SCD or by cause other than SCD. Interested patients/families meeting inclusion and exclusion eligibility criteria will be signing a written assent/consent.

Study Protocol:

20 patients meeting eligibility criteria will receive standard treatment antalgic fluids (isotonic sodium chloride solution, 10 mL/kg over 30 minutes) and morphine if needed. Patients of group 1 who continued to meet eligibility criteria after completion of emergency department evaluation and standard treatment will receive a Vascular Electrical Stimulation Therapy (VEST) treatment for 4 hours placing two electrodes under each calf and two electrodes under each arm at clinical dose (40 to 50 volts). The VEST treatment can be repeated during 3 days if the vaso-occlusive crisis persists.

Pain assessment, blood pressure determination, oxygen saturation (measured by pulse oximetry), and laboratory studies will be performed immediately prior to VEST treatment, each hour during the 4 hours of treatment, and for 6 hours after treatment. The amount of parenteral narcotic used during the first 24 hours will be recorded. Antalgic and fluids will be the only medications allowed between the time that patients entered the emergency department until after the 10-hours duration of the VEST treatment/observation period.

Criteria for early termination from the study include, oxygen saturation as measured by pulse oximetry (SpO2) of less than 90%, mean systolic blood pressure of less than 80 mm Hg, increase in pain score of more than 2 cm during the VEST treatment period, and patient/family request for termination.

Outcome Measures:

According to US Department of Health and Human Services guidelines on the management of SCD, self-reporting of pain is the most reliable indicator of the presence and intensity of pain. The 10-cm visual analog scale (VAS) is an easy to use, reliable pain assessment tool in patients older than 5 years and has been extensively validated in patients with SCD.

The primary outcome measure is the change in pain score every hour during the first 10 hours and the time requested for managing the vaso-occlusive crisis in the patient. The primary pain assessment tool is a 10-cm horizontal VAS labelled with “0” to correspond to no pain at one end and “10” to indicate worst pain at the other. The VAS test will be administered by the same investigator in each site throughout the study using standardized instructions. The vaso-occlusive crisis is considered as managed when the pain score falls and stays below 2 cm in visual analog scale during at least 6 consecutive hours. Secondary outcomes include change in pain over time, amount of morphine administered to the patient, change in anaemia over 24 hours, duration of acute vaso-occlusive crisis (corresponding to a pain score of at least 4 cm in VAS) and length of hospitalization. Safety assessments included minimum systolic blood pressure and minimum SpO2.

Data Analysis:

With a sample size of 20 patients (10 per group), the study is 80% power to detect a mean difference of at least 2.0 cm in the change in VAS pain score between groups at 4 hours of treatment compared with immediately prior to treatment, using an unpaired 2-tailed t test with a 0.05 significance level assuming a common SD of 1.5 cm. The study is not powered to detect differences in secondary outcome measures. The study will be monitored for safety throughout by an independent data and safety monitoring board. A formal interim analysis to evaluate study safety and potential early evidence of efficacy is planned and will be conducted after approximately 50% of patients will complete the study.

Changes in pain score will be compared using an unpaired 2-tailed t test. Baseline characteristics and secondary outcome measures will be compared using unpaired t tests for continuous variables and the Fisher exact test for categorical variables. All statistical analyses were carried out using SPSS, version 9.0 (SPSS Inc, Chicago, Ill.) and S-PLUS 2000 (Insightful Corp, Seattle, Wash.).

### Example 5: Interventional, Prospective, Randomized, Comparative, Single Blind and Mono-Centric Study to Evaluate the Efficacy of VEST in the Treatment of Vaso-Occlusive Crises (VOC) in Paediatric Patients

The protocol of this study is similar to that provided in Example 4 except that paediatric patients of at least 4 year-old are enrolled.

### Example 6: Clinical Study Concerning the Assessment of VEST in Combination with NSAID in the Relief of Pain in Vaso-Occlusive Crisis Associated with Sickle Cell Disease

The objective of the clinical study was to assess the efficacy of VEST in combination with non-steroid anti-inflammatory drugs to manage pain in adult and paediatric patients experimenting a severe vaso-occlusive crisis associated with sickle cell disease.

27 patients suffering from a sickle-cell disease and experimenting a severe vaso-occlusive crisis, with a pain score of at least 6 in the visual analog scale for pain (VAS pain), were enrolled in the study. The enrolled patients comprised 16 adults (of 19 to 35 years) and 11 children (of 8 to 17 years). 8 patients were of HbSS genotype, 19 patients were of HbSC genotype. The enrolled patients comprised 18 male subjects and 9 female subjects.

After their admission at the hospital, 15 patients received pain killer (Perfalgan) and (Tramadol) drugs and the NSAID Diclofenac and were subjected to VEST treatment for 4 hours, with two electrodes under each calf and two electrodes under each arm of the patient.

2 patients received Pain killer (Perfalgan) and (Tramadol) drug and were subjected to VEST treatment for 4 hours, with two electrodes under each calf and two electrodes under each arm of the patient. These patients were not administered with any NSAID.

10 patients received pain killer (Perfalgan) and (Tramadol) drugs and Diclofenac only without being subjected to VEST.

Pain change in each patient was assessed with a 10-cm visual analog scale (VAS) for pain (e.g. as shown in FIG. 4) every two hours, during 6 hours after the beginning of VEST.

Results

The results of the study are shown in FIG. 5. VEST treatment was effective to reduce pain in all patients but the pain reduction was significantly higher in the group treated with NSAID in combination with VEST. The mean pain score in VAS was decreased by 60% within two hours after the beginning of VEST associated with NSAID. The mean pain score even fell below 2 cm (mild pain) after 4 hours of VEST+NSAID treatment. In patients treated with VEST administrated without NSAID the VAS score was reduced to 4 cm after 4 hours. On the other hand, treatment with NSAID alone without VEST reduced the VAS score to 6 cm only.

After 6 hours, the mean pain score fell at 0.6 cm in patients treated with VEST and NSAID treatment while in patients treated with VEST the VAS score was reduced to 2.5 cm and in patients treated with NSAID alone the VAS score remained at 6 cm.

In conclusion, all patients treated with VEST had a significant pain reduction as compared to patients treated with NSAID alone. This reduction was significantly higher and faster when the VEST treatment was associated with NSAID drug, which suggests a synergic action of the combination VEST-NSAID.

1-26. (canceled)

27. A method for treating or delaying the onset of a vaso-occlusive crisis in a patient suffering from a hemoglobinopathy which comprises subjecting the patient to vascular electrical stimulation therapy (VEST).

28. The method of claim 27, wherein the hemoglobinopathy is selected from the group consisting of a sickle-cell disease, HbS/HbE syndrome, HbA/HbS genotype, HbE/β⁺ thalassaemia, and HbE/β⁺ thalassaemia.

29. The method of claim 28, wherein the patient suffers from a sickle-cell disease selected from the group consisting of sickle-cell anemia, HbSC disease, HbS/β⁰ thalassaemia, and HbS/β⁺ thalassaemia.

30. The method of claim 27, wherein the VEST comprises administering the patient with an electrical periodic current having a frequency from 0.01 Hz to 15 Hz.

31. The method of claim 30, wherein the electrical periodic current has a frequency from 0.4 Hz to 9 Hz.

32. The method of claim 30, wherein the periodic current is composed of electric pulses, the duration of each pulse being of at least 0.5 ms with a rise duration (T₁) of at least 0.25 ms and a decrease duration (T₂) of at least 0.25 ms.

33. The method of claim 30, wherein the electrical periodic current administered to the patient is composed of unidirectional positive pulses, unidirectional negative pulses or streams of pulses which are alternatively negative or positive.

34. The method of claim 30, wherein the electrical periodic current administered to the patient is composed of pulses and has at least one of the following features:
the extinction duration (T₁) of the pulse is from 0.2 to 5-fold the rise duration (T₂) of the pulse,
the rise duration (T₁) is of at least 0.25 ms and the decrease duration (T₂) is of at least 0.25 ms,
the peak amplitude of the pulse is from −130 V to 130 V,
the base width of the pulse is from 0.5 ms to 30 ms, and
the period of the electrical current is from 100 ms to 5000 ms.

35. The method of claim 30, wherein the pulses of the electrical periodic current have an exponential-type waveform.

36. The method of claim 30, wherein the electrical periodic current is administered to the patient transcutaneously.

37. The method of claim 30, wherein the electrical periodic current is administered to the patient by a medical device for electrotherapy, comprising at least two electrodes disposed on the skin of the patient.

38. The method of claim 37, wherein the medical device for electrotherapy comprises at least four electrodes and wherein at least one electrode is disposed on each wrist and each calf of the patients.

39. The method of claim 27, wherein the patient is subjected to vascular electrical stimulation therapy after being rehydrated and/or after being administered with drug(s) selected from analgesics.

40. The method of claim 27, wherein the method further comprises administering an anti-inflammatory drug (NSAID) to the patient.

41. The method of claim 27, wherein the patient is subjected to one to four VEST sessions daily, each VEST session lasting from about 10 min to about one hour.

42. A method for improving the general condition of patient suffering from a hemoglobinopathy which comprises subjecting the patient to a long-term treatment with VEST.

43. The method of claim 42, wherein VEST is chronically administered to the subject transcutaneously for at least 3 months.

44. The method of claim 43, wherein the subject suffers from a sickle-cell disease and the symptom is selected from the group consisting of anemia, chronic pain, chronic fatigue, joint pain, rheumatism, breathlessness, priapism, hand-foot syndrome, splenic sequestration, and combinations thereof.

45. A method for treating or delaying the onset of one or several symptoms associated with a hemoglobinopathy in a patient, which comprises subjecting the subject to a long-term treatment with VEST.

46. The method of claim 45, wherein VEST is chronically administered to the subject transcutaneously for at least 3 months.
